(19) United States
(12) Patent Application Publication
(10) Pub. No.: US 2017/0087029 A1
(43) Pub. Date: Mar. 30, 2017

(54) METHOD OF MAKING BREATHABLE, ELASTIC FILM LAMINATES AND ARTICLES DERIVED THEREFROM

(71) Applicant: 3M INNOVATIVE PROPERTIES COMPANY, St. Paul, MN (US)

(72) Inventors: Todd L. Nelson, Eau Claire, WI (US); Robert E. Andrews, Sheboygan Falls, WI (US); William H. Sikorski, Jr., Birchwood Village, MN (US)

(21) Appl. No.: 15/308,543

(22) PCT Filed: Apr. 27, 2015
(86) PCT No.: PCT/US2015/027822

§ 371 Date: Nov. 2, 2016

Related U.S. Application Data
(60) Provisional application No. 61/987,919, filed on May 2, 2014.

(51) Int. Cl. A61F 13/15; B32B 27/32; B32B 5/02; B32B 27/12; A61F 13/49; B32B 3/26

(52) U.S. Cl. CPC A61F 13/15593; A61F 13/4902; A61F 13/15699; B32B 3/266; B32B 5/022; B32B 27/12; B32B 27/32; A61F 2013/49023; B32B 2262/0253; B32B 2307/51; B32B 2307/724; B32B 2307/7265; B32B 2555/02

(57) ABSTRACT

A method of making a breathable, elastic film laminate comprising the steps of stretching an elastic film in a first direction, spot welding at least one nonwoven web to the film while it is stretched, and stretching the film laminate in a second direction to create apertures in the film. Articles derived from the method are also described.

### FIELD OF INVENTION

The present disclosure relates to a method of making breathable, elastic film laminates and the breathable, elastic film laminates derived therefrom. The present disclosure also relates to the use of such breathable, elastic film laminates in personal care products, such as diapers, training pants, adult incontinence devices, booties and garments.

### BACKGROUND

Elastic films are commonly incorporated into personal care products to better shape the products to the contours of the body. Elastic films can be used, for example, in the waist and leg areas of diapers, the side panels of training pants, and the cuffs of disposable gowns. Since elastic films tend to be somewhat tacky, it is common to apply one or more web layers, such as a nonwoven layer, to the elastic films to improve processing and feel. Typically, a nonwoven layer is joined to the elastic film in a stretched state. When the elastic film is allowed to recover, the nonwoven layer gathers, or shirrs, to create an elastic film laminate in the direction of stretch.

One of the disadvantages of such laminates is the lack of breathability. Although the web layers are often air permeable, the films are typically not. Methods that enhance breathability have particular value in the personal care industry where both conformity and comfort are important.

### SUMMARY

The present disclosure relates to breathable, elastic film laminates.

In one embodiment, the disclosure provides a method of making a breathable, elastic film laminate comprising providing an elastic film having a first surface and a second surface opposite the first surface, stretching the film in a first direction, spot welding a nonwoven web to the first surface of the film while it is stretched in the first direction to produce a film laminate with multiple weld sites, and stretching the film laminate in a second direction to create apertures in the film.

In another embodiment, the disclosure provides an article comprising an elastic film having a first surface and a second surface opposite the first surface, a nonwoven web laminated to the first surface of the film at multiple weld sites, and apertures in the elastic film associated with at least some of the weld sites, each aperture extending outward from the periphery of a single weld site in predominately a single direction.

As used herein, the term “spot welding,” and variations thereof, refer to bonding two or more materials together in a small area, or spot, by the application of heat and/or pressure. Spot welding methods include ultrasonic welding, heated embossing, laser welding and high pressure welding.

As used herein, the terms “activate,”“activation,” and variations thereof, refer to the process by which a material has been mechanically deformed so as to impart elasticity to a least a portion of the material.

As used herein, the term “recover,” and variations thereof, refers to the contraction of a stretched material upon termination of a biasing force following stretching of the material by application of the biasing force.

As used herein, the term “machine direction,” or “MD,” refers to the direction of a running, continuous film and/or web during the manufacture of a film laminate.

As used herein, the term “cross direction,” or “CD,” refers to the direction which is essentially perpendicular to the machine direction.

As used herein, the terms “including,” “comprising,” or “having” and variations thereof encompass the items listed thereafter and equivalents thereof, as well as additional items.

As used herein, the terms “first,” “second,” and the like are only used to describe elements as they relate to one another, and are in no way meant to recite specific orientations of an article or apparatus, to indicate or imply necessary or required orientations of an article or apparatus, or to specify how an article or apparatus described herein will be used, mounted, displayed, or positioned in use.

All numerical ranges are inclusive of their endpoints and non-integral values between the endpoints unless otherwise stated.

The above summary of the present disclosure is not intended to describe each disclosed embodiment or every implementation of the present disclosure. The description that follows more particularly exemplifies illustrative embodiments. It is to be understood, therefore, that the drawings and following description are for illustrative purposes only and should not be read in a manner that would unduly limit the scope of this disclosure.

### BRIEF DESCRIPTION OF THE DRAWINGS

FIG. 1 is a schematic view of an exemplary apparatus used to carry out a method of making breathable, elastic film laminates;

FIG. 2 is a schematic view of an alternative exemplary apparatus used to carry out a method of making breathable, elastic film laminates;

FIG. 3A is a schematic view of one embodiment of a breathable, elastic film laminate;

FIG. 3B is a schematic view of a second embodiment of a breathable, elastic film laminate;

FIG. 4A is a photomicrograph of an exemplary breathable, elastic film laminate;

FIG. 4B is a photomicrograph of a single aperture in an exemplary breathable, elastic film laminate;

FIG. 5 is a photomicrograph of another exemplary breathable, elastic film laminate;

FIG. 6 is a schematic view of an adult incontinence device; and

FIGS. 7A-C are schematic views of an exemplary method of making an adult incontinence device containing a breathable, elastic film laminate.

### DETAILED DESCRIPTION

The present disclosure relates to a method of making breathable, elastic film laminates (hereinafter, also referred to as “breathable laminate”) from an elastic film and at least one nonwoven web. Suitable films and webs will be described in further detail below. However, the nonwoven webs are typically air permeable whereas the films are not. The present disclosure describes how to combine these two materials into a film laminate that is not only elastic but also provides sufficient breathability for a variety of applications, including personal care applications.

Generally, the method includes providing an elastic film having a first surface and a second surface opposite the first surface and stretching the film in a first direction (i.e., pre-lamination stretch). First direction is a relative term that in the context of large scale production can include machine direction, cross direction and any direction in-between. For ease of processing, however, the first direction is typically the machine direction. The amount of stretch in the first direction will depend to some extent on the nature of the film and desired elasticity of the finished breathable laminate. In some embodiments, the film may be stretched at least 200% (i.e., stretched to three times the original length), more particularly at least 250%. Typically, the film can be stretched up to the point of mechanical failure, such as tearing.

While the film is in the stretched state, a nonwoven web is spot welded to the first surface of the film to produce a film laminate with multiple weld sites. Optionally, a second nonwoven web may be welded to the second surface of the film. The welding is sufficient enough to fragment the film in the region of the weld sites but not strong enough to produce a hole all the way through the nonwoven web(s). In some embodiments, where the film laminate comprises a nonwoven web on each side of the film, the two nonwoven webs may be directly bonded to each other through the weld sites. The air permeability at each of the weld sites increases relative to the rest of the film laminate due to the fragmentation of the film. However, the amount of air passage is typically not enough to satisfy the breathability requirements of many applications, including personal care articles.

It has been found, however, that breathability can be enhanced by further stretching the spot welded film laminate (i.e., post-lamination stretch) in a second direction to create apertures (i.e., openings) in the film. The additional stress placed on the laminate causes the film to tear at the periphery of the weld site. That tear then propagates outward from the periphery of the weld site in predominately a single direction to create an aperture through which air can pass. The apertures in the film are large enough to create the desired breathability in the film laminate but not so large that they contact more than one weld site (e.g., an aperture will not bridge two weld sites). The nonwoven web(s) remains intact over the weld sites and apertures, thus the integrity of the nonwoven web(s) is maintained.

The second direction of the post-lamination stretch can be the same or different from the first direction of the pre-lamination stretch. For processing convenience, the first and second directions are both typically in the machine direction. The amount of post-lamination stretch should be sufficient to create apertures but not so great that an aperture bridges two or more weld sites. The post-lamination stretch should also be less than the film break point. In some embodiments, the film laminate may be stretched at least 0.9%, more particularly at least 1.5% beyond its position during lamination.

Although typically the film laminate is not recovered between the lamination and post-lamination stretch, this need not be the case. The film laminate can be relaxed, either fully or partially, after lamination and prior to the post-lamination stretch.

After the post-lamination stretch, the breathable laminate may be recovered, either immediately or at a later time.

Generally, the breathable laminates produced by the above method comprise an elastic film having a first surface and a second surface opposite the first surface. A nonwoven web is laminated to the first surface of the film at multiple weld sites. Alternatively, a second nonwoven web is laminated to the second surface of the film. Apertures in the film are associated with at least some weld sites. Each aperture extends outward from the periphery of a single weld site in predominately a single direction.

Although it is preferable that an aperture be associated with each weld site, this is not required. Tears may initiate at some weld sites but not others. In some embodiments, apertures are associated with at least 50%, more particularly at least at least 60%, and even more particularly at least 70% of the weld sites. Moreover, those apertures that form, may do so to different degrees (i.e., some variation in aperture shape and size). In some embodiments, the average area of the individual apertures ranges from about 0.10 mm² to about 0.61 mm², more particularly about 0.28 mm² to about 0.61 mm². Furthermore, some weld sites may have multiple apertures. For example, during the post-lamination stretch, tears may originate on opposite sides of a weld site, creating an aperture that extends outward from the periphery of the weld site in one direction and a second aperture that extends outward from the periphery of the weld site in the opposite direction.

The number of apertures and size of the apertures will affect the breathability of the film laminate. Therefore, one measure of breathability is the percentage of the area of apertures to the total film area. For personal care articles, it is preferable that the apertures in the breathable laminates of the present disclosure make up at least 1.8%, more particularly 3.4%, even more particularly 5.3% of the total film area.

A second measure of breathability is the pressure drop across the breathable laminate. For personal care articles, it is preferable that the breathable laminate exhibits an average pressure drop of 6 mm H₂O or less, more particularly 4 mm H₂O or less, or even more particularly 2 mm H₂O or less at a flow rate of 50 liters/minute when stretched 100% in the first direction.

Although the above method describes spot welding as a means to create breathable laminates, it is also possible to apply additional spot welding to the film laminate for the purpose of reducing the overall elasticity of the breathable laminate and/or enhancing the integrity of the breathable laminate. This can be done, for example, by taking the film laminate after the post-lamination stretch, partially recovering the film laminate, and spot welding the partially recovered film laminate to create additional weld sites. The film laminate is typically recovered to the point at which the laminate existed during the lamination step. Alternatively, the film laminate can be fully recovered and subsequently re-stretched. Since spot welding over an aperture can reduce the breathability of that aperture, it is preferable that any second (third, fourth, etc) generation of weld sites be sufficiently offset from the original weld sites to minimize impact to breathability.

Films suitable for use in the above method exhibit elastomeric properties at ambient conditions, i.e. the films will substantially resume their original shape after being stretched. The films may comprise a single layer or multiple layers. The films may be elastic, whether inherently or as the result of an activation step. Elastic films include those that are activated prior to, or during, the above described method. For example, a relatively inelastic film that is activated during the pre-lamination stretch in the method of the present disclosure is an elastic film for the purposes of this disclosure. The terms “elastomeric” and “elastic” refer to the same properties and are used interchangeably throughout.

In some exemplary embodiments, the film can be made from either pure elastomers or blends with an elastomeric phase or content that will still exhibit substantial elastomeric properties at room temperature. Suitable thermoplastic elastomers include block copolymers or the like. Particularly useful block copolymers include styrene/isoprene, butadiene or ethylene-butylene/styrene block copolymers. Generally, the block copolymers contain an A block and a B block. These blocks may be arranged in any order including linear, radial, branched or star block copolymers. Other useful elastomeric compositions can include elastomeric polyurethanes, ethylene copolymers such as ethylene vinyl acetates, ethylene/propylene copolymer elastomers or ethylene/propylene/diene terpolymer elastomers. Blends of these elastomers with each other or with modifying elastomers are also contemplated.

Viscosity reducing polymers and plasticizers can also be blended with the elastomers such as low molecular weight polyethylene and polypropylene polymers and copolymers, or tackifying resins. Examples of tackifiers include aliphatic or aromatic hydrocarbon liquid tackifiers, polyterpene resin tackifiers, and hydrogenated tackifying resins.

Additives such as dyes, pigments, antioxidants, antistatic agents, bonding aids, fillers, antiblocking agents, slip agents, heat stabilizers, photostabilizers, foaming agents, glass bubbles, reinforcing fiber, starch and metal salts for degradability or microfibers can also be used in the elastomeric core layer.

The films can be made by a variety of methods, including extrusion, co-extrusion, solvent casting, foaming, and the like. In a preferred embodiment, the film is a multilayer film comprising two relatively inelastic skin layers and an elastomeric core layer sandwiched therebetween. The multilayer film is relatively inelastic prior to activation. However, the film can be rendered elastic by stretching the multilayer film past the elastic deformation limit of the skin layers to produce a multilayer film that is elastic in the direction of stretch. Due to the deformation of the skin layers during activation, the multilayer film exhibits a microtextured surface upon recovery. Microtexture refers to the structure of the skin layers in the area of activation. More particularly, the skin layers contain peak and valley irregularities or folds, the details of which cannot be seen without magnification.

The elastomeric core layer can broadly include any material which is capable of being formed into a thin film layer and exhibits elastomeric properties at ambient conditions. Preferably, the elastomeric core layer will sustain only small permanent set following deformation of the skin layers and recovery, which set is preferably less than 20 percent and more preferably less than 10 percent of the original length after moderate elongation, e.g., about 100-200%. Generally, any elastomeric core layer is acceptable which is capable of being stretched to a degree that causes relatively consistent permanent deformation in the skin layers. This can be as low as 50% elongation. Preferably, however, the elastomeric core layer is capable of undergoing up to 300% to 800% elongation at room temperature. The elastomeric core layer can be both pure elastomers and blends with an elastomeric phase or content that will still exhibit substantial elastomeric properties at room temperature.

Both heat-shrink and non-heat-shrinkable elastomers are contemplated for use in the present invention. However, non-heat-shrinkable elastomers are preferred from a processing standpoint. Non-heat-shrinkable means that the elastomer, when stretched, will substantially recover without application of heat, sustaining only a small permanent set as discussed above. Non-heat-shrinkable polymers include block copolymers such as those known to those skilled in the art as A-B or A-B-A block copolymers. These block copolymers are described, for example, in U.S. Pat. No. 3,265,765, “Block Polymers of Monovinyl Aromatic Hydrocarbons and Conjugated Dienes,” (Holden, et al.); U.S. Pat. No. 3,562,356, “Block Copolymer Blends with Certain Ethylene-Unsaturated Ester Copolymers,” (Nyberg, et al.); U.S. Pat. No. 3,700,633, “Selectively Hyrdogenated Block Copolymers,” (Wald, et al.); U.S. Pat. No. 4,116,917, “Hydrogenated Star-Shaped Polymer,” (Eckert); and U.S. Pat. No. 4,156,673, “Hydrogenated Star-Shaped Polymer,” (Eckert). Styrene/isoprene, butadiene or ethylene-butylene/styrene (SIS, SBS or SEBS) block copolymers are particularly useful. Other useful elastomeric compositions can include elastomeric polyurethanes, ethylene copolymers such as ethylene vinyl acetates, ethylene/propylene copolymer elastomers or ethylene/propylene/diene terpolymer elastomers. Blends of these elastomers with each other or with modifying non-elastomers are also contemplated. In some embodiments, the elastomeric core layer is a blend of styrene-isoprene-styrene (SIS) and polystyrene. In more particular embodiments, the SIS:polystyrene weight ratio ranges from 2:1 to 19:1.

The same viscosity reducing polymers and plasticizers, tackifiers and additives mentioned above may be blended with the elastomers of the core layer.

The skin layers can be formed of any semi-crystalline or amorphous polymer that is less elastic than the elastomeric core layer and will undergo permanent deformation at the desired percent stretch of the multilayer film. Therefore, slightly elastomeric compounds, such as some olefinic elastomers, e.g. ethylene-propylene elastomers or ethylene-propylene-diene terpolymer elastomers or ethylenic copolymers, e.g., ethylene vinyl acetate, can be used as skin layers, either alone or in blends. However, the skin layer is generally a polyolefin such as polyethylene, polypropylene, polybutylene or a polyethylene-polypropylene copolymer, but may also be wholly or partly polyamide such as nylon, polyester such as polyethylene terephthalate, polyvinylidene, polyacrylate such as poly(methyl methacrylate) (only in blends) and the like, and blends thereof.

Additives useful in the skin layers include, but are not limited to, mineral oil extenders, antistatic agents, pigments, dyes, antiblocking agents, provided in amounts less than about 15%, starch and metal salts for degradability and stabilizers.

Other layers may be added between the elastomeric core layer and the skin layers, such as tie layers, to improve the bonding of the skin and core layers. Tie layers can be formed of, or compounded with, for example, maleic anhydride modified elastomers, ethyl vinyl acetates and olefins, polyacrylic imides, butyl acrylates, peroxides such as peroxypolymers, e.g., peroxyolefins, silanes, e.g., epoxysilanes, reactive polystyrenes, chlorinated polyethylene, acrylic acid modified polyolefins and ethyl vinyl acetates with acetate and anhydride functional groups and the like, which can also be used in blends or as compatibilizers or delamination-promoting additives in one or more of the skin or core layers.

The multilayer films can be prepared by coextrusion of the elastomeric core layer and skin layers. Alternatively, the multilayer films can be prepared by application of the elastomeric core layer onto the skin layers or vice versa. Such techniques are well-known to those skilled in the art.

The core:skin thickness ratio of the multilayer films are preferably controlled to allow for an essentially homogeneous activation of the multilayer film. The core:skin thickness ratio is defined as the ratio of the thickness of the elastomeric core layer over the sum of the thicknesses of the two skin layers. Additionally, the core:skin thickness ratio of the multilayer film needs to be selected so that when the skin layers are stretched beyond their elastic deformation limit and relaxed with the elastomeric core layer, the skin layers form a microtextured surface. The desired core:skin ratio will depend upon several factors, including the composition of the film. In some embodiments of the present invention, the core:skin ratio of the multilayer film is at least 2:1. In other embodiments, the core:skin ratio of the multilayer film is at least 3:1.

The skin layers of the multilayer films may be the same composition or different. Similarly, the skin layers may be the same thickness or different. In one preferred embodiment, the skin layers are the same composition and thickness.

In some embodiments of the present invention, the core layer of the multilayer film is a styrenic block copolymer and the skin layers of the multilayer film are each a polyolefin. In other embodiments, the core layer of the multilayer film is a SIS and polystyrene blend and the skin layers of the multilayer film are each a polypropylene and polyethylene blend. In yet other embodiments, the core layer of the multilayer film is a SIS and polystyrene blend and the skin layers of the multilayer film are each polypropylene.

Exemplary multilayer films for the present invention are disclosed in U.S. Pat. No. 5,462,708, “Elastic Film Laminate,” (Swenson, et al.), U.S. Pat. No. 5,344,691, “Spatially Modified Elastic Laminates,” (Hanschen, et al.), and U.S. Pat. No. 5,501,679, “Elastomeric Laminates with Microtextured Skin Layers,” (Krueger, et al.), which are incorporated herein by reference. Suitable commercially available films include M-340 Pant Elastic, available from 3M Company of St. Paul, Minn., USA.

The composition of the nonwoven web used in the above method is not particularly limiting. The term “nonwoven web” generally refers to a web having a structure of individual fibers or threads which are interlaid, but not in an identifiable manner, as in a knitted fabric. Exemplary nonwoven webs include spunbond webs, carded webs, dry laid webs, meltblown webs and combinations thereof. The webs can be elastic or inelastic. The nonwoven webs typically have a basis weight ranging from 8 gsm to 20 gsm. The fibers making up the nonwoven webs typically have a fiber size ranging from 1.5 denier to 8 denier, more particularly from 1.8 denier to 4 denier.

Spunbond nonwoven webs are made by extruding a molten thermoplastic, as filaments, from a series of fine die orifices in a spinneret. The diameter of the extruded filaments is rapidly reduced under tension by, for example, non-eductive or eductive fluid-drawing or other known spunbond mechanisms, such as described in U.S. Pat. No. 4,340,563, “Method of Forming Nonwoven Webs,” (Appel et al.); U.S. Pat. No. 3,692,618, “Continuous Filament Nonwoven Web,” (Dorschner et al.); U.S. Pat. No. 3,338,992, “Process for Forming Non-Woven Filamentary Structures from Fiber-Forming Synthetic Organic polymers,” and U.S. Pat. No. 3,341,394, “Sheets of Randomly Distributed Continuous Filaments,” (Kinney); U.S. Pat. No. 3,276,944, “Non-Woven Sheet of Synthetic Organic Polymeric Filaments and Method of Preparing Same,” (Levy); U.S. Pat. No. 3,502,538, “Bonded Nonwoven Sheets with a Defined Distribution of Bond Strengths,” (Peterson); U.S. Pat. No. 3,502,763, “Process of Producing Non-Woven Fabric Fleece,” (Hartman) and U.S. Pat. No. 3,542,615, “Process for Producing a Nylon Non-Woven Fabric,” (Dobo et al.). The spunbond web is preferably bonded (e.g., point or continuous bonded).

The nonwoven web may also be made from carded webs. Carded webs are made from separated staple fibers that are sent through a combing or carding unit which separates and aligns the staple fibers in the machine direction so as to form a generally machine direction-oriented fibrous nonwoven web. However, randomizers can be used to reduce this machine direction orientation.

Once the carded web has been formed, it is typically bonded by one or more of several bonding methods to give it suitable tensile properties. One bonding method is powder bonding wherein a powdered adhesive is distributed through the web and then activated, usually by heating the web and adhesive with hot air. Another bonding method is pattern bonding wherein heated calender rolls or ultrasonic bonding equipment are used to bond the fibers together, usually in a localized bond pattern though the web can be bonded across its entire surface if so desired. Generally, the more the fibers of a web are bonded together, the greater the nonwoven web tensile properties.

Airlaying is another process by which fibrous nonwoven webs can be made. In the airlaying process, bundles of small fibers usually having lengths ranging between 6 to 19 millimeters are separated and entrained in an air supply and then deposited onto a forming screen, often with the assistance of a vacuum supply. The randomly deposited fibers are then bonded to one another using, for example, hot air or a spray adhesive.

Meltblown nonwoven webs may be formed by extrusion of thermoplastic polymers from multiple die orifices, where the polymer melt streams are immediately attenuated by hot high velocity air or steam along two faces of the die immediately at the location where the polymer exits from the die orifices. The resulting fibers are entangled into a coherent web in the resulting turbulent airstream prior to collection on a collecting surface. Meltblown webs may be further bonded such as by through air bonding, heat or ultrasonic bonding.

Nonwoven webs may be made of synthetic fibers (e.g., thermoplastic fibers) or a combination of synthetic fibers and natural fibers (e.g., wood, cotton or wool). Exemplary materials for forming thermoplastic fibers include polyolefins, polyamides, polyesters, copolymers containing acrylic monomers, and blends and copolymers thereof. Suitable polyolefins include polyethylene, e.g., linear low density polyethylene, high density polyethylene, low density polyethylene and medium density polyethylene; polypropylene, e.g., isotactic polypropylene, syndiotactic polypropylene, blends thereof and blends of isotactic polypropylene and atactic polypropylene; and polybutylene, e.g., poly(1-butene) and poly(2-butene); polypentene, e.g., poly-4-methylpentene-1 and poly(2-pentene); as well as blends and copolymers thereof. Suitable polyamides include nylon 6, nylon 6/6, nylon 10, nylon 4/6, nylon 10/10, nylon 12, nylon 6/12, nylon 12/12, and hydrophilic polyamide copolymers such as copolymers of caprolactam and an alkylene oxide, e.g., ethylene oxide, and copolymers of hexamethylene adipamide and an alkylene oxide, as well as blends and copolymers thereof. Suitable polyesters include polyethylene terephthalate, polybutylene terephthalate, polycyclohexylenedimethylene terephthalate, and blends and copolymers thereof. Acrylic copolymers include ethylene acrylic acid, ethylene methacrylic acid, ethylene methylacrylate, ethylene ethylacrylate, ethylene butylacrylate and blends thereof. Particularly suitable polymers are polyolefins, including polyethylene, e.g., linear low density polyethylene, low density polyethylene, medium density polyethylene, high density polyethylene and blends thereof; polypropylene; polybutylene; and copolymers as well as blends thereof.

The nonwoven webs may be made from a single component fiber, a bicomponent fiber, or combinations thereof. The term “bicomponent”, as used herein, means comprising two or more separate components, each of which extends longitudinally along the fiber through a cross-sectional area of the fiber. For example, in a fiber comprising two components, the first component may be disposed more in the center of the fiber, with the second component wrapped partially or completely around the first component. In the latter case, the first component becomes a core and the second component becomes a sheath. More than two different polymeric materials may be included in bicomponent fibers, e.g., as separate layers.

Bicomponent fibers may be formed from a wide variety of fiber-forming materials. Representative combinations of polymeric materials for the components of a fiber include: polyester (e.g., polyethylene terephthalate) and polypropylene; polyethylene and polypropylene; polyester (e.g, polyethylene terephthalate) and linear polyamides such as nylon 6; polybutylene and polypropylene; and polystyrene and polypropylene. Also, different materials may be blended to serve as one component of a bicomponent fiber.

The nonwoven webs of the present disclosure may be made of a single fiber or blends of two or more fibers having, for example, different compositions, diameters and/or lengths. The composition of the web may be uniform throughout or vary, for example, within the web. The nonwoven webs of the present disclosure may be made of a single layer or multiple layers. In some embodiments, the nonwoven web is a spunbond-spunbond-spunbond (SSS) layered nonwoven web. A suitable commercial SSS nonwoven web is Fitesa Product Number S16Q1KR1AAQ1A, available from Fitesa of Simpsonville, S.C., USA.

The nonwoven webs may also include additional ingredients, such as dyes, pigments, binders, bleaching agents, thickening agents, softening agents, detergents, surface active agents, and combinations thereof.

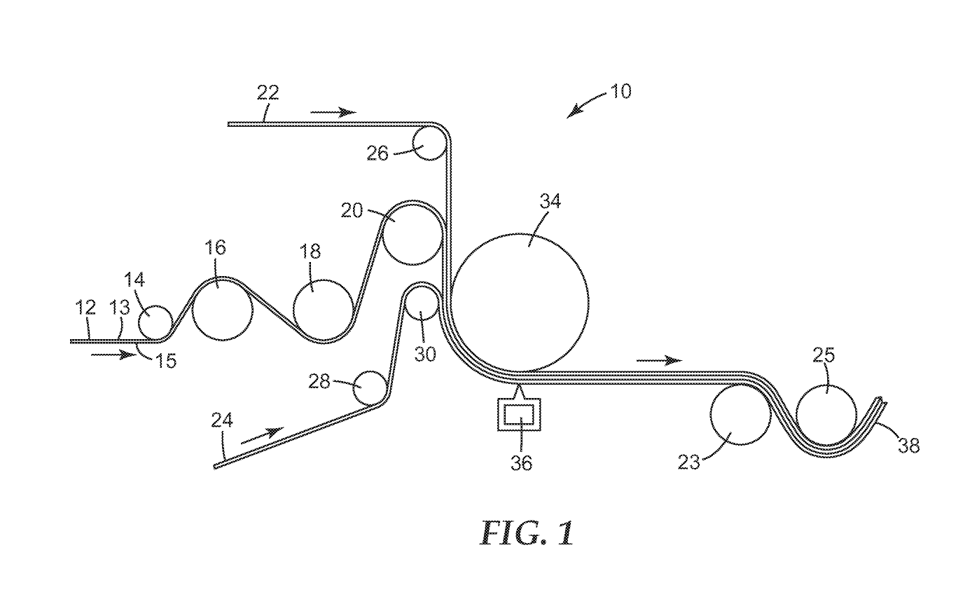

FIG. 1 illustrates an exemplary apparatus 10 that can be used to carry out the method of the present disclosure. A suitable commercially available apparatus that includes a stretch bonding station is a J8-T Adult Pant Machine, available from Curt G. Joa, Inc. of Sheboygan Falls, Wisc., USA. An elastic film 12 having a first surface 13 and a second surface 15 opposite the first surface 13 is unwound from a supply roll (not shown) and transferred by one or more guide rolls 14 to a series of differential speed rolls 16, 18, 20. As the film 12 passes through the differential speed rolls 16, 18, 20, the film is stretched in a first direction (in this case the MD).

In one embodiment, the differential speed rolls 16, 18, 20 operate at increasingly greater speeds the further downstream they are located, with roll 20 operating at the greatest speed and roll 16 operating at the lowest speed. The speed may increase linearly or non-linearly from one roll to the next. In an alternative embodiment, the speed rolls 16, 18, 20 may pulsate. For example, roll 18 may operate at a slower speed than either of rolls 16 and 20, causing the film to go through sequences of stretch and recovery. The distance between adjacent speed rolls 16, 18, 20 can be the same or different. Although three differential speed rolls 16, 18, 20 are illustrated in FIG. 1, it should be understood that two or more differential speed rolls may be used.

The amount of stretch in the first direction will depend to some extent on the nature of the film and desired extensibility of the finished film laminate. In some embodiments, the film may be stretched at least 200%, more particularly at least 250%.

Nonwoven web 22 is unwound from a supply roll (not shown) and transferred by guide roll 26 to the first surface 13 of the film 12. Nonwoven web 24 is similarly unwound from a supply roll (not shown) and transferred by guide roll 28 to the second surface 15 of the film 12. Although the nonwovens webs 22, 24 are typically the same dimensions in the CD, it not need be the case. In some embodiments, the nonwoven webs 22, 24 are applied to only a portion of the film 12. In other embodiments, the nonwoven webs 22, 24 are coextensive with the film 12. In yet other embodiments, the nonwoven webs 22, 24 are wider in the CD than the film 12. The nonwoven webs 22, 24 can be the same composition or different.

The MD stretched film 12 and nonwoven webs 22, 24 are then laminated together using any of a variety of spot welding techniques (e.g., ultrasonic welding, heated embossing, laser welding and high pressure welding). Ultrasonic welding is illustrated by way of example in FIG. 1. Ultrasonic welding generally refers to a process performed, for example, by passing the film 12 and nonwoven webs 22, 24 between a sonic horn 36 and a patterned roll (e.g., anvil roll) 34. Such welding methods are well-known in the art. For instance, ultrasonic welding through the use of a stationary horn and a rotating patterned anvil roll is described in U.S. Pat. No. 3,844,869, “Apparatus for Ultrasonic Welding of Sheet Materials,” (Rust Jr.); and U.S. Pat. No. 4,259,399, “Ultrasonic Nonwoven Bonding,” (Hill). Moreover, ultrasonic welding through the use of a rotary horn with a rotating patterned anvil roll is described in U.S. Pat. No. 5,096,532, “Ultrasonic Rotary Horn,” (Neuwirth, et al.); U.S. Pat. No. 5,110,403, “High Efficiency Ultrasonic Rotary Horn,” (Ehlert); and U.S. Pat. No. 5,817,199, “Methods and Apparatus for a Full Width Ultrasonic Bonding Device,” (Brennecke, et al.). Of course, any other ultrasonic welding technique may also be used in the present disclosure.

In some embodiments, the patterned roll 34 and differential speed roll 20 operate at the same speed. In alternative embodiments, the patterned roll 34 and differential speed roll 20 operate at different speeds, where the patterned roll 34 acts as an extension of the differential speed rolls 16, 18, 20.

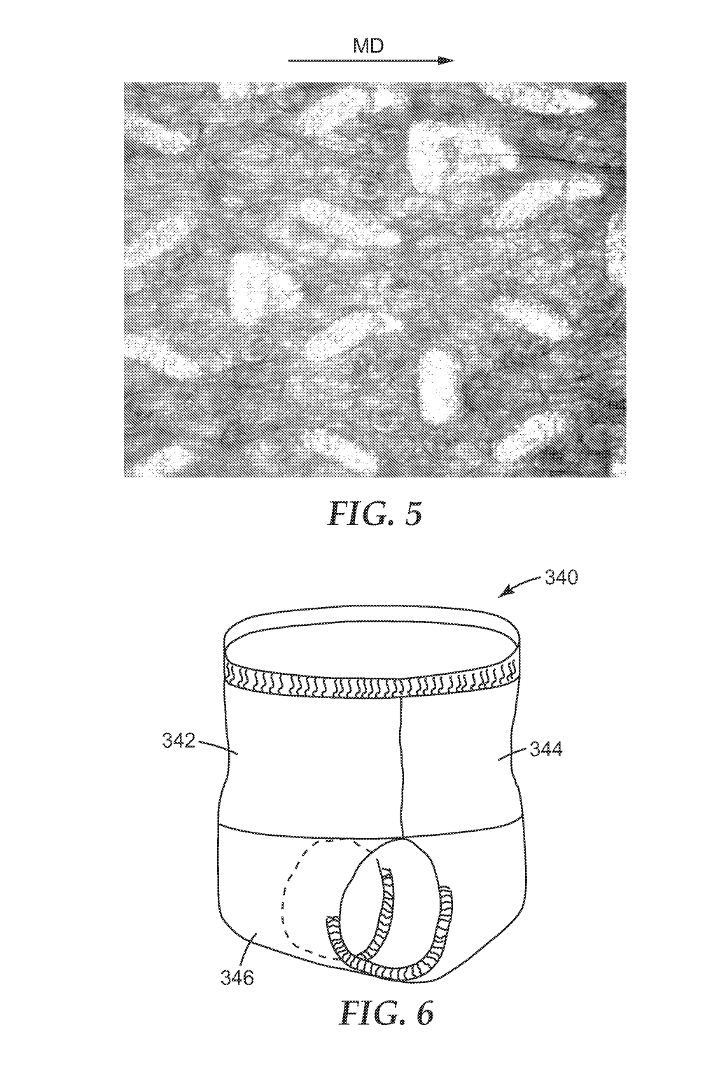

The pattern of roll 34 is not particularly limiting. Exemplary patterns may include one or more elements, such as circles, ovals, squares, rectangles, triangles, polygons or combinations thereof. The elements within a pattern may be the same or different sizes. The elements may be arranged randomly, in a repeating pattern, or combination thereof. In some embodiments, the pattern is a repeating arrangement of circles. In other embodiments, the pattern is a group of ovals arranged in star configuration. A breathable laminate made using a pattern roll having such a star configuration is shown in FIG. 5.

The sonically welded film 12 and nonwoven webs 22, 24 are withdrawn from the pattern roll 34 as a film laminate and further stretched in a second direction (in this case the MD) by the differential speed rolls 23, 25 to create apertures. In some embodiments, the film laminate may be stretched at least 0.9%, more particularly at least 1.5%.

The film laminate 38 is then wound onto a storage roll (not shown) for incorporation into a product in a separate process. The film laminate 38 may be allowed to recover before winding on the storage roll, or the film laminate can be wound onto the storage roll in a stretched state and recovered at a later time. Alternatively, the film laminate 38 may be fed directly to a manufacturing line for finished goods. For example, the film laminate may be maintained in a stretched state after it is withdrawn from the pattern roll 34 and incorporated directly into a product in a downstream process before allowing the film laminate to recover.

Although the film laminate 38 in FIG. 1 has two nonwoven webs, film laminates with only one nonwoven web may be produced by simply eliminating one of the webs 22, 24 from the process. In cases where the film laminate comprises a single nonwoven web, it may be preferable to use an activatable multilayer film having an elastic core layer in-between two relatively inelastic skin layers. The resultant film laminate will have a soft nonwoven web on one side and microtextured film surface on the other side. The microtextured surface is typically non-tacky and soft to the touch, and can be used as an external layer in various processes and applications.

The apparatus shown in FIG. 1 uses differential speed rolls to stretch the film and laminate in the MD. However, the apparatus could also be set up to stretch the film and/or laminate in the CD by replacing the differential speed rolls with any of number of well known CD stretching devices including, but not limited to, tenter frames, diverging disks, and incremental stretching devices. Stretching by tenter frames is described, for instance, in U.S. Pat. No. 7,320,948, “Extensible Laminate Having Improved Stretch Properties and Method for Making Same,” (Morman, et al.). Stretching by diverging disks is described, for example, in U.S. Publication 2011/0151739, “Activatable Precursor of a Composite Laminate Web and Elastic Composite Laminate Web,” (Bosler et al.). A suitable incremental stretching device includes the ring-rolling apparatus described in U.S. Pat. No. 5,366,782, “Polymeric Web Having Deformed Sections Which Provide a Substantially Increased Elasticity to the Web,” (Curro). In alternative embodiments, a combination of differential speed rolls and CD stretching devices can be used in concert to stretch either the film and/or laminate in directions that lie between MD and CD.

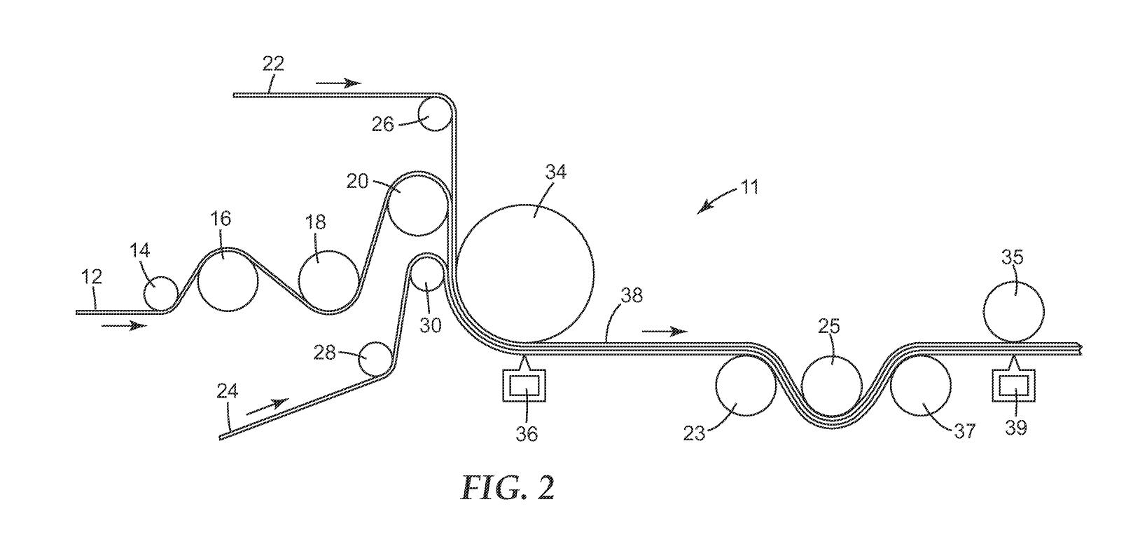

In some instances, it may be desirable to create additional welding within the breathable laminate of the present disclosure for purposes other than breathability. For example, weld sites can reduce elasticity and/or enhance the integrity of a film laminate. FIG. 2 illustrates a modification to the apparatus 10 in FIG. 1 that provides for such additional welding.

The apparatus 11 in FIG. 2 differs from the apparatus 10 in FIG. 1 in that the film laminate is withdrawn from the differential speed roll 25, partially recovered as it is fed over guide roll 37, and passed through a second sonic horn 39 and patterned roll 35 to provide additional weld sites. The weld sites produced by sonic horn 39 are preferably off-set from those created by sonic horn 36 in order to minimize any impact on the breathability of the film laminate. Partially recovered in this context means that at a minimum the tension on the film laminate is the same as when it exited the patterned roll 34 but less than the tension applied by differential speed rolls 23, 25. In an alternative embodiment, the film laminate may be fully recovered and subsequently stretched the appropriate amount before the second ultrasonic welding step. In such a case, it would not be necessary to have two ultrasonic welding stations. The film laminate could be fed back through the apparatus in FIG. 1 a second time.

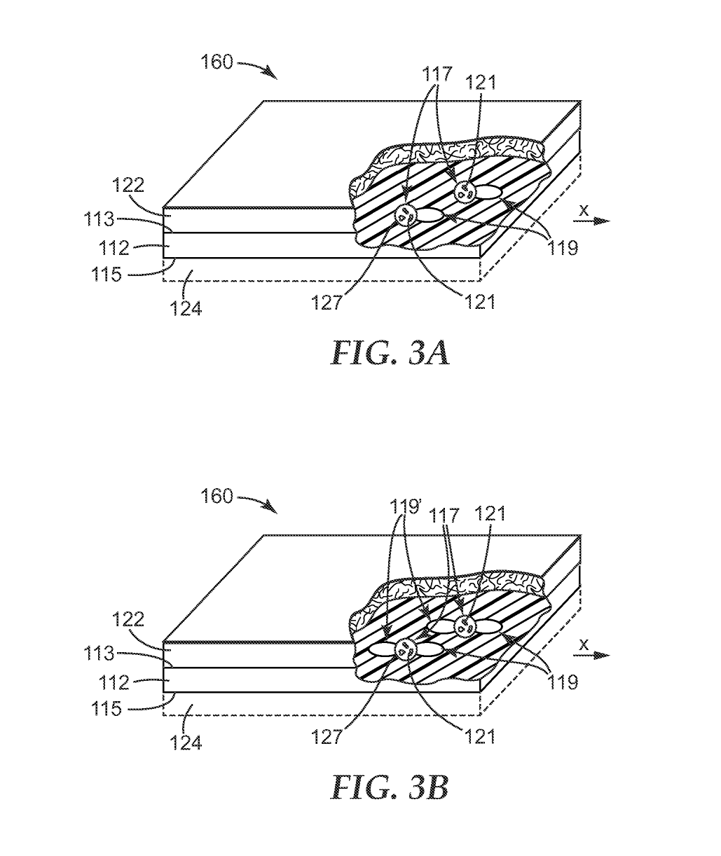

An exemplary breathable laminate 160 produced by the method of the present disclosure is illustrated in FIG. 3A. The film laminate 160 comprises an elastic film 112 having a first surface 113 and a second surface 115 opposite the first surface 113. A nonwoven web 122 is laminated to the first surface 113 of the film 112 at multiple weld sites 117. Optionally, a second nonwoven web 124 is laminated to the second surface 115 of the film 112 at the multiple weld sites 117. Apertures 119 in the film 112 are associated with at least some of the weld sites 117, each aperture 119 adjoining the periphery 127 of a single weld site and extending outward from the periphery 127 in predominately a single direction “x”.

The nonwoven webs 122, 124 extend across the entire film laminate. Although there may be variations in basis weight and loft across the webs 122, 124, particularly over the weld sites 117, the nonwoven webs 122, 124 remain essentially intact. In contrast, the film may be reduce to fragments 121 in the weld sites 117 and completely absent from the apertures 119. Without wishing to be bound by theory, it is believed that the film at the periphery 121 of the weld site 117 is weakened relative to the rest of the film. Thus, further stretching of the film laminate initiates a tear at the periphery 127 of the weld site 117. The tear then propagates predominately in a single direction “x”. In some embodiments, tears will initiate on opposite sides of the weld site such that one aperture extends outward from the periphery of the weld site in one direction and a second aperture extends outward from the periphery of the weld site in the opposite direction. Such a second aperture 119′ is illustrated in FIG. 3B.

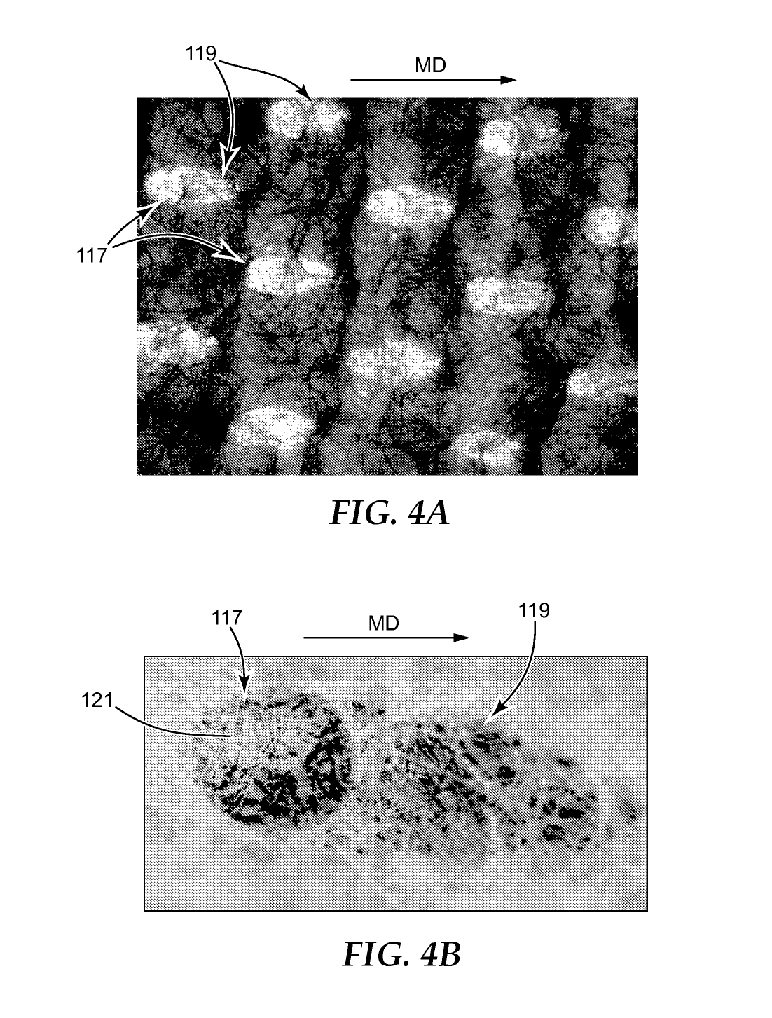

FIGS. 4A-4B are photomicrographs of exemplary breathable laminates made according the disclosed method. The breathable laminate comprises two nonwoven webs and a film therebetween. In FIG. 4A, a number of weld sites 117 and apertures 119 are visible. FIG. 4B is a close-up of a single weld site 117 and aperture 119. The weld sites 117 contain fragments 121 of film bonded to the nonwoven webs. The apertures 119 are free of film.

The shape and location of the weld sites are reflective of the pattern of elements on the patterned roll used to spot weld the nonwoven webs to the film. The weld sites in FIGS. 4A-4B are evenly spaced and relatively circular, reflecting a patterned roll where the surface is a regular array of circles. The shapes of the weld sites are limited only by the ability to machine the surface of a patterned roll. In some embodiments, the shapes of the weld sites are circular, oval, square, rectangular, triangular, polygonal, or combinations thereof. In some preferable embodiments, the weld sites are circular. Typically, the weld sites are far enough apart so that when the apertures are created, they do not adjoin more than one weld site.

The breathable, elastic film laminates made according to the above method can be used in a variety of applications. Suitable applications include, but are not limited to, elastic components in personal care products such as diapers, training pants, adult incontinence devices, booties and garments.

FIG. 6 illustrates an adult incontinence device 340 comprising breathable laminates of the present disclosure. The adult incontinence device 340 comprises a front waist region 342, back waist region 344 and central region 346.

During use, the central region 346 fits between a user's legs and is designed to absorb and retain bodily fluids. The central region typically comprises a liquid permeable topsheet, a liquid impermeable backsheet and an absorbent core enclosed therebetween. The liquid permeable topsheet may consist of a nonwoven web, such as already described above with respect to the nonwoven webs in the breathable laminates. Further examples of topsheet materials are porous foams, and apertured plastic films. The materials suitable as topsheet materials should be soft and non-irritating to the skin and be readily penetrated by urine.

The liquid impermeable backsheet may consist of a thin plastic film, e.g., a polyethylene or polypropylene film, a nonwoven material coated with a liquid impervious material, a hydrophobic nonwoven material which resists liquid penetration, or laminates of plastic films and nonwoven materials. The backsheet material may be breathable so as to allow vapor to escape from the absorbent core, while still preventing liquids from passing through the backsheet material.

The topsheet and the backsheet material typically extend beyond the absorbent core and are connected to each other, e.g., by gluing or welding by heat or ultrasonic, about the periphery of the absorbent core. The topsheet and/or the backsheet may further be attached to the absorbent core by any method known in the art, such as adhesive, heat bonding etc. The absorbent core may also be unattached to the topsheet and/or the backsheet.

The absorbent body can be of any conventional kind. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbents in an absorbent body. It is also common to have absorbent bodies comprising layers of different material with different properties with respect to liquid receiving capacity, liquid distribution capacity and storage capacity. The thin absorbent bodies often comprise a compressed mixed or layered structure of cellulosic fluff pulp and superabsorbent.

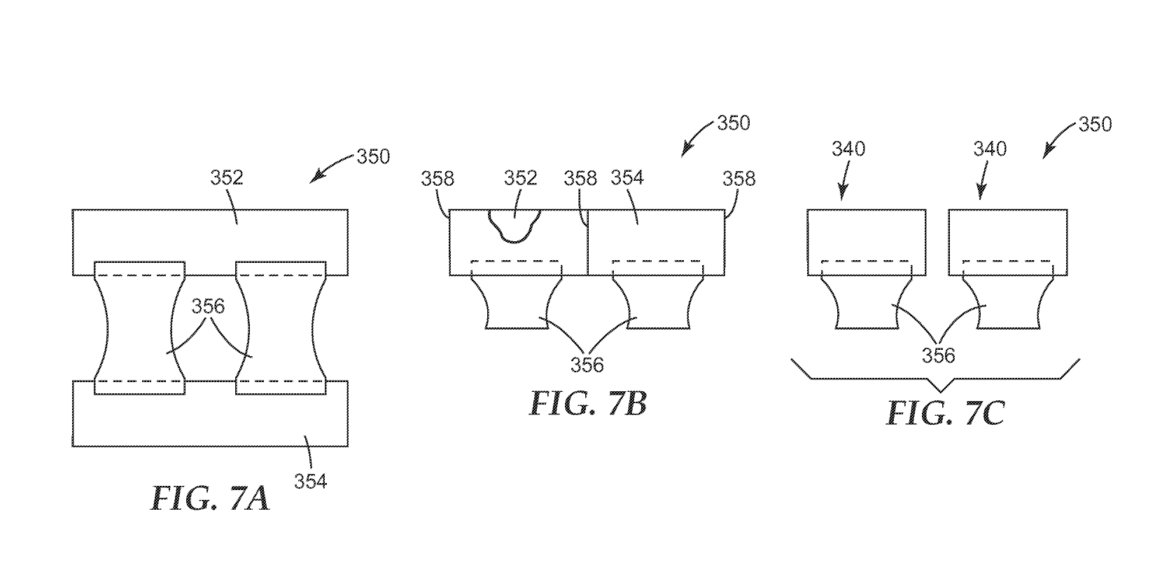

A process 350 for making the adult incontinence device 340 is illustrated in FIGS. 7A-C. The front and back waist regions 342, 344 in FIG. 6 are made from the breathable laminates of the present disclosure and assist in conforming the adult incontinent device 340 to the contours of the body and providing added comfort through breathability.

As illustrated in FIG. 7A, two breathable laminates 352, 354 of the present disclosure are run parallel to each other on a manufacturing line 350. One breathable laminate corresponds to the front waist region 342 of the adult incontinence device and the second breathable laminate corresponds to the back waist region 344, as depicted in FIG. 6. The breathable laminates 352, 354 are typically maintained in a stretched state during processing. A gap exists between the two breathable laminates for placement of the central region 356 of the adult incontinence device.

The central region 356 typically comprises a liquid permeable topsheet, a liquid impermeable backsheet and an absorbent core enclosed therebetween, as discussed above. The central region can be assembled off-line or assembled further upstream in the process 350. Either way, the central region 356 is laid across the breathable laminates 352, 354 such that one end of the central region 356 overlaps breathable laminate 352, and the opposing end of the central region 356 overlaps breathable laminate 354. Central regions 356 are laid down at predetermined intervals, leaving a gap between adjacent central regions 356. The central regions 356 are attached to the breathable laminates 352, 354 using any number of known techniques including, but not limited to, adhesive bonding, heat bonding, ultrasonic welding, sewing or the like.

The central regions 356 may be transferred to the breathable laminates 352, 354 using traditional mechanical methods. However, because the breathable laminates allow for sufficient passage of air, the central regions 356 can be applied to the breathable laminates 352, 354 using vacuum transfer. Vacuum transfer allows for faster processing rates and more precise alignment of the central regions 356 across the breathable laminates 352, 354.

The assembled web (i.e., breathable laminates 352, 354 and central regions 356 ) is then folded over onto itself as illustrated in FIG. 7b such that the two breathable laminates are coextensive with each other. The breathable laminates 352, 354 are then attached along bond lines 358 by, e.g., gluing or welding by heat or ultrasonic, and simultaneously, or subsequently severed. The breathable laminates 352, 354 recover to create adult incontinence devices 340, as illustrated in FIGS. 7C and 6.

FIGS. 7A-C illustrate just one method for making articles containing the breathable laminates of the present disclosure. There are numerous variations on this method that are within the knowledge of one skilled in the art. Moreover, the breathable laminates of the present disclosure can be used in a variety of applications where elastics are typically used to conform articles to the contour of the body. Methods for making such articles are also well-known.

### Some Embodiments of the Disclosure

In a first embodiment, the present disclosure provides a method of making a breathable, elastic film laminate comprising providing an elastic film having a first surface and a second surface opposite the first surface, stretching the film in a first direction, spot welding a nonwoven web to the first surface of the film while it is stretched in the first direction to produce a film laminate with multiple weld site, and stretching the film laminate in a second direction to create apertures in the film.

In a second embodiment, the present disclosure provides the method of the first embodiment, wherein each aperture extends outward from the periphery of a single weld site in predominately a single direction.

In a third embodiment, the present disclosure provides the method of the first or second embodiment, wherein two apertures extend outward from the periphery of a single weld site in opposite directions.

In a fourth embodiment, the present disclosure provides the method of any one of the first to third embodiments, further comprising the addition of a second nonwoven web to the second surface of the film during the spot welding step.

In a fifth embodiment, the present disclosure provides the method of any one of the first to fourth embodiments, wherein the film is a multilayer film.

In a sixth embodiment, the present disclosure provides the method of any one of the first to fifth embodiments, wherein the film comprises two skin layers and an elastomeric core layer sandwiched therebetween.

In a seventh embodiment, the present disclosure provides the method of the sixth embodiment, wherein the skin layers comprise polypropylene.

In an eighth embodiment, the present disclosure provides the method of any one of the first to seventh embodiments, wherein the nonwoven web comprises polypropylene.

In a ninth embodiment, the present disclosure provides the method of any one of the first to eighth embodiments, wherein the film is stretched at least 200% in the first direction.

In a tenth embodiment, the present disclosure provides the method of any one of the first to ninth embodiments, wherein the film laminate is stretched at least 0.9% in the second direction.

In an eleventh embodiment, the present disclosure provides the method of any one of the first to tenth embodiments, wherein the first and second directions are the same.

In a twelfth embodiment, the present disclosure provides the method of any one of the first to eleventh embodiments, wherein the first and second directions are each in the machine direction.

In a thirteenth embodiment, the present disclosure provides the method of any one of the first to tenth embodiments, wherein the first and second directions are different.

In a fourteenth embodiment, the present disclosure provides the method of any one of the first to thirteenth embodiments, wherein the average area of the individual apertures ranges from about 0.10 mm² to about 0.61 mm².

In a fifteenth embodiment, the present disclosure provides the method of any one of the first to fourteenth embodiments, wherein apertures are associated with at least 50% of the weld sites.

In a sixteenth embodiment, the present disclosure provides the method of any one of the first to fifteenth embodiments, wherein the apertures make up at least 1.8% of the total elastic film area.

In a seventeenth embodiment, the present disclosure provides the method of any one of the first to sixteenth embodiments, wherein the breathable, elastic film laminate exhibits an average pressure drop of 6 mm H₂O or less at a flow rate of 50 liters/minute when stretched 100% in the machine direction.

In an eighteenth embodiment, the present disclosure provides the method of any one of the first to seventeenth embodiments, further comprising the steps of partially recovering the film laminate, and spot welding the partially recovered film laminate to create additional weld sites.

In a nineteenth embodiment, the present disclosure provides an article comprising an elastic film having a first surface and a second surface opposite the first surface, a nonwoven web laminated to the first surface of the film at multiple weld sites, and apertures in the elastic film associated with at least some of the weld sites, each aperture extending outward from the periphery of a single weld site in predominately a single direction.

In a twentieth embodiment, the present disclosure provides the article of the nineteenth embodiment, further comprising a second nonwoven web laminated to the second surface of the film at the multiple weld sites.

In a twenty-first embodiment, the present disclosure provides the article of any the nineteenth or twentieth embodiment, wherein the elastic film is a multilayer film.

In a twenty-second embodiment, the present disclosure provides the article of any one of the nineteenth to twenty-first embodiments, wherein the elastic film comprises two skin layers and an elastomeric core layer sandwiched therebetween.

In a twenty-third embodiment, the present disclosure provides the article of the twenty-second embodiment, wherein the skin layers comprise polypropylene.

In a twenty-fourth embodiment, the present disclosure provides the article of any one of the nineteenth to twenty-third embodiments, wherein the nonwoven web comprises polypropylene.

In a twenty-fifth embodiment, the present disclosure provides the article of any one of the nineteenth to twenty-fourth embodiments, wherein the weld sites comprise fragments of elastic film welded to the nonwoven web.

In a twenty-sixth embodiment, the present disclosure provides the article of any one of the nineteenth to twenty-fifth embodiments, wherein the shapes of the weld sites are selected from the list consisting of circular, oval, square, rectangular, triangular, polygonal, and combinations thereof.

In a twenty-seventh embodiment, the present disclosure provides the article of any one of the nineteenth to twenty-sixth embodiments, wherein the shapes of the weld sites are circular.

In a twenty-eighth embodiment, the present disclosure provides the article of any one of the nineteenth to twenty-seventh embodiments, wherein the average area of the individual apertures ranges from about 0.10 mm² to about 0.61 mm².

In a twenty-ninth embodiment, the present disclosure provides the article of any one of the nineteenth to twenty-eighth embodiments, wherein apertures are associated with at least 50% of the weld sites.

In a thirtieth embodiment, the present disclosure provides the article of any one of the nineteenth to twenty-ninth embodiments, wherein the apertures make up at least 1.8% of the total elastic film area.

In a thirty-first embodiment, the present disclosure provides the article of any one of the nineteenth to thirtieth embodiments, wherein the breathable, elastic film laminate exhibits an average pressure drop of 6 mm H₂O or less at a flow rate of 50 liters/minute when stretched 100% in the machine direction.

In a thirty-second embodiment, the present disclosure provides a personal care article comprising any one of the nineteenth to thirty-first embodiments,

### EXAMPLES

The following examples are presented to illustrate some breathable, elastic film laminates of the present disclosure and are not intended in any way to otherwise limit the scope of the invention.

### Materials

M-340 Pant Elastic—a trilayer film laminate having a styrenic block copolymer elastic core sandwiched between two polyolefin inelastic skin layers (available from 3M Company of St. Paul, Minn., USA). Fitesa Product Number S16Q1KR1AAQ1A—a 16 gsm spunbond-spunbond-spunbond nonwoven web (available from Fitesa of Simpsonville, S.C., USA).

### Pressure Drop Test

A TSI Model 8130 Automated Filter Test Instrument (available from TSI® Incorporated of Shoreview, Minn., USA) was calibrated using a steel orifice plate. The aerosol was turned off, and the pressure drop was measure at 50 liters/minute in mm H₂O. All samples were stretch 100% in the MD.

The TSI Model 8130 Instrument uses a standard 4.5 inch (114.3 mm) diameter circular sample to measure the pressure drop through a surface area of 10261 mm². However, the laminate samples were only 71 mm diameter with a pressure drop through a surface area of 3959 mm². Therefore, it was necessary to develop a correlation that accounted for the sample size differences. Assuming that the pressure drop versus surface area is linear at a given flow rate, the ratio of the areas was taken into consideration for pressure drop calculations. The pressure drop obtained from the instrument at 50 liters/minute was divided by 2.59 (the ratio of area for 10261 mm² to 3959 mm²) to get the standard measurements in mm H₂O.

Five measurements were taken for each sample and the average reported in TABLE 1.

### Average Apertured Area

A Keyence VHX-600 Microscope (available from Keyence Corporation of America of Elmwood Park, N.J., USA) at 50× magnification was used to measure the area of individual apertures in the film laminates. The polygon tool was used to outline each aperture and determine the actual area. All measurements were made when the film laminate sample was stretched 100% in the MD. The area of five randomly selected apertures in the center of the sample was measured and the average area of individual apertures determined.

An Image-Pro Microscope (Version 7.0 ) (also available from Keyence) at 7.1× magnification was used to determine the number of apertures in a given area of the same film laminate. The film laminate was stretch 100% in the MD and measurements were taken from the center of the film laminate. The number of apertures in an 80 mm² area of the sample was measured at five random locations and the average number of apertures in 80 mm² determined.

The apertured area was calculated according to the following formula:

(average number of apertures in 80 mm² area)×(average area of individual apertures)×100% (80 mm²)

The results are reported in TABLE 1.

### Samples E1-E5 and C1-C4

All samples were made according to the method illustrated in FIG. 1 using the stretch bonding station of a J8-T Adult Pant Machine available from Curt G. Joa, Inc. of Sheboygan Falls, Wisc., USA.

A Fitesa S16Q1KR1AAQ1A nonwoven web was applied to each side of a M-340 Pant Elastic film that had been stretched 300% in the machine direction. The film and webs were sonically welded to each other at the speeds and pressures provided in TABLE 1 The sonically welded film was further stretched (i.e., post-lamination stretch) by the amount also provided in TABLE 1.

The average pressure drop and open area were measured for each sample and the results provided in TABLE 1. For the purposes of personal care articles, it is desirable that the film laminate exhibit an average pressure drop of 6 mm H₂O or less at a flow rate of 50 liters/minute when stretched 100% in the machine direction. Those samples meeting the desired pressure drop are designated by the letter “E”.

TABLE 1

| | Sonic | Anvil Roll | Post- | Ave Pressure | Apertured |
|---|---|---|---|---|---|
| | Pressure | Speed | Lamination | Drop (per | Area |
| Sample | (PSI) | (surface fpm)* | Stretch (%) | mm H₂O) | (%) |
| E1 | 31 | 300 | 1.7 | 2.2 | 7.1 |
| C1 | 31 | 300 | 0.3 | 7.1 | 1.1 |
| E2 | 28 | 1049 | 1.5 | 3.3 | 3.4 |
| C2 | 28 | 1054 | 0.3 | 11.7 | 0.6 |
| E3 | 28 | 300 | 1.7 | 2.8 | 5.3 |
| C3 | 28 | 300 | 0.3 | 15.5 | 0.6 |
| E4 | 31 | 1049 | 1.5 | 4.1 | 3.4 |
| C4 | 31 | 1054 | 0.3 | 11.4 | 0.6 |
| E5 | 30 | 676 | 0.9 | 4.3 | 2.0 |

*Feet per minute.

The embodiments described above and illustrated in the figures are presented by way of example only and are not intended as a limitation upon the concepts and principles of the present invention.

Thus, the invention provides, among other things, a method of making breathable, elastic film laminates and articles derived therefrom. Various features and advantages are set forth in the following claims.

1. A method of making a breathable, elastic film laminate comprising:
providing an elastic film having a first surface and a second surface opposite the first surface;
stretching the film in a first direction;
spot welding a nonwoven web to the first surface of the film while it is stretched in the first direction to produce a film laminate with multiple weld sites; and
stretching the film laminate in a second direction to create apertures in the film.

2. The method of claim 1, wherein each aperture extends outward from the periphery of a single weld site in predominately a single direction.

3. The method of claim 1, wherein two apertures extend outward from the periphery of a single weld site in opposite directions.

4. The method of claim 1, further comprising the addition of a second nonwoven web to the second surface of the film during the spot welding step.

5. The method of claim 1, wherein the film is a multilayer film.

6. The method of claim 1, wherein the film comprises two skin layers and an elastomeric core layer sandwiched therebetween.

7. The method of claim 6, wherein the skin layers comprise polypropylene.

8. The method of claim 1, wherein the nonwoven web comprises polypropylene.

9. The method of claim 1, wherein the film is stretched at least 200% in the first direction.

10. The method of claim 1, wherein the film laminate is stretched at least 0.9% in the second direction.

11. The method of claim 1, wherein the first and second directions are the same.

12. The method of claim 1, wherein the first and second directions are each in the machine direction.

13. The method of claim 1, wherein the first and second directions are different.

14. The method of claim 1, wherein the average area of the individual apertures ranges from about 0.10 mm² to about 0.61 mm².

15. The method of claim 1, wherein apertures are associated with at least 50% of the weld sites.

16. The method of claim 1, wherein the apertures make up at least 1.8% of the total elastic film area.

17. The method of claim 1, wherein the breathable, elastic film laminate exhibits an average pressure drop of 6 mm H₂O or less at a flow rate of 50 liters/minute when stretched 100% in the machine direction.

18. The method of claim 1, further comprising the steps of partially recovering the film laminate, and spot welding the partially recovered film laminate to create additional weld sites.

19.-32. (canceled)
